(19) United States
(12) Patent Application Publication
(10) Pub. No.: US 2021/0145589 A1
(43) Pub. Date: May 20, 2021

(54) BONE AUGMENTATION PIECE AND KIT CONSISTING OF A BONE AUGMENTATION PIECE WITH AN INSERTED (DENTAL) IMPLANT

(71) Applicant: KARL LEIBINGER MEDIZINTECHNIK GMBH & CO. KG, Mühlheim (DE)

(72) Inventors: Martin RÜCKER, Zürich (CH); Harald ESSIG, Zürich (CH); Frank REINAUER, Emmingen-Liptingen (DE); Adem AKSU, VS-Schwenningen (DE)

(21) Appl. No.: 16/629,701

(22) PCT Filed: Jul. 10, 2018
(86) PCT No.: PCT/EP2018/068664

§ 371 Date: Jan. 9, 2020

(30) Foreign Application Priority Data
Jul. 1, 2017 (DE) 10 2017 115 404.3

(51) Int. Cl. A61F 2/30; A61F 2/28

(52) U.S. Cl. CPC A61F 2/30734; A61F 2/2803; A61F 2002/30062; A61F 2002/30578; A61F 2002/30784; A61B 17/176; A61F 2310/00179; A61F 2310/00011; A61F 2002/30153; A61F 2002/30156; A61F 2002/30914

(57) ABSTRACT

The invention relates to a bone augmentation piece (1) for filling out a defective area (2) in a bone (3), comprising a main part (5) which is designed to be inserted into said defective area (2) without a gap, wherein the main part (5) comprises a truss-type structure made of webs (6) and cavities (7). The invention also relates to a kit consisting of a bone augmentation piece (1) of this type, comprising an implant inserted thereinto, such as a dental implant. The invention also relates to the use of the claimed bone augmentation piece (1) as a drill jig (18).

The invention relates to a bone augmentation piece for filling out a defective area, such as a hole, a slit or a gap, in a bone, such as a maxilla or a mandible, but outside the field of application of dysgnathia.

From the state of the art already numerous bone implants have been known but so far not those which fill out a defective area in a bone as a bone augmentation piece. Usually, plates are applied to the outside of the bone to fix the hole or, resp., the defective area in position, and subsequently bone filling material, such as granulate, is introduced to the defective area. However, said granulate always has the drawback that it is not dimensionally stable per se. The individual granulated parts must be inserted painstakingly into the defective area, must be compacted there so as to consolidate there over time only.

US 2017/014 550 A1 discloses an implantable scaffold matrix for bone regeneration, which is made of a biodegradable polymer material. In addition, U.S. Pat. No. 6,214,049 B1 and U.S. Pat. No. 6,077,076 A each disclose different lattice-like or fibrous structures with a biodegradable layer, which are used to surround an implant placed in the bone and promote bone regeneration. Furthermore, US 2006/052873 A1 discloses a manufacturing process for an implant having a fibrous or knitted structure of resorbable material.

However, especially faster healing and better handling for the operating surgeon are intended to be achieved. Also, a cost-efficient bone implant which grows in quickly and ensures proper surgical success is intended to be provided.

When bone is lost in the area of the teeth-bearing alveolar ridge, for example within the framework of accidents, resections with benign or malignant tumors, inflammations and further indications, usually a reconstruction of the bony alveolar ridge (augmentation) and later insertion of dental implants is indicated. Accordingly, a common procedure is the autogenous bone transplantation. To this end, the bone is regionally removed (e.g. in the middle part of the face/in the part of the mandible which does not bear any teeth), especially from the ala of the ilium. In order to prevent a successive donor defect and morbidity in the donor area, alloplastic granulates are increasingly used for augmentation of the alveolar ridge. Said granulates are not dimensionally stable, however, and must be protected against undesired ingrowth of connective tissue by additional membranes. Recently there have been developed 3D titanium meshes which address said drawback of the missing dimensional stability but must be completely removed again prior to a planned dental implantation.

The autogenous bone transplantation has the drawbacks of collecting morbidity in the donor area, requirement of complicated contouring to the defective situation and limitation as to the local availability of the bone. Alloplastic granulates are not dimensionally stable and require membranes, as they have no membrane characteristic per se and do not enable any integrated 3D planning Regrettably, 3D titanium meshes are not resorbable, exhibit no membrane characteristic, either, and must necessarily be removed prior to implantation. A plurality of interventions is especially undesired, however.

Therefore, it is the object of the present invention to eliminate said drawbacks.

According to a first aspect of the invention there is provided a bone augmentation piece for filling out a defective area in a bone, comprising a main part which is adapted to be inserted into the defective area without a gap, wherein the main part comprises a truss-type structure made of webs and cavities which define meshes, contains or is made up from biodegradable metallic materials and/or resorbable polymers or ceramic materials, and the webs and meshes are surrounded on the outside by a rim, the rim having more than five times the thickness of a web.

Said object is achieved, according to the invention, by the fact that the bone augmentation piece for filling out/filling in a defective area is provided with a main part which is adapted to be inserted into the defective area without a gap (as far as possible), wherein the solid or macro-/micro-porous main part has a truss-type structure made of webs/strands which in turn can be designed to be solid or macro-/micro-porous and made of cavities defining meshes so as to be dimensionally stable, that the main part contains or is made up from biodegradable metallic materials and/or resorbable polymers or ceramic materials, and that the webs and meshes are surrounded on the outside by a rim, the rim having more than five times the thickness of a web. Said initial dimensional stability is of help in introducing the bone augmentation piece.

Moreover, the truss-type structure made from a material which is biodegradable helps, on the one hand, to support faster osteogenesis by bone material growing into the implant and, on the other hand, to produce an almost original and, resp., natural structure, as the implant will degrade and thus only the bone material will remain within the body. Equally, the grid-type structure helps to degrade the foreign material, as a large surface contact is realized between the bone material and the implant.

Advantageous embodiments are claimed in the subclaims and shall be detailed below.

It is of advantage when plural webs are intersecting and the webs are interconnected at the intersection point, preferably in one piece/one material there so that they are connected at the intersection forming an integral component.

It is useful when the webs form a grid structure. In so doing, meshes are formed. All or most of said meshes then are intended to have an (approximately or exactly) equal surface area. In this way, ingrowth can be exactly controlled and predicted. Then the grid structure is grown through especially evenly by regenerative bone material, thus entailing proper surgical success.

For manufacture it is advantageous when the meshes take a triangular shape, especially that of an equilateral or isosceles triangle, or when the meshes take a rectangular shape, especially that of an equilateral rectangle/square. Then the arrangement of the webs/strands can be especially properly predetermined, which is beneficial to manufacture.

The initial dimensional stability becomes especially efficiently determinable/designable when the webs, e.g. all webs or the majority of the webs, are equal in terms of geometry and/or concerning the material properties.

It is of advantage when the webs have a linear/straight or corrugated or curved design. Also, a linear design in portions having an upstream and/or downstream transition to a corrugated/curved shape is advantageous. It has also proven itself when constantly the same curvature is given in designing a curved shape. In this context, it is worth mentioning that a curvature increasing when viewed over the length or, alternatively, a decreasing curvature may offer advantages. Also, twisting of the webs is imaginable.

An advantageous example embodiment is also characterized in that the webs and meshes define a wall that is in the form of a pocket. For example, an opening can be provided at the bone augmentation piece/the bone implant, e.g. on the upper or lower side, to receive fills such as bone slurry/bone chips.

It is of advantage when the main part includes holes/cavities of from 10 μm to 700 μm. When a porosity is provided, air inclusions of average extensions of from 10 μm to 200 μm have proven themselves. The pocket is provided to be filled with allographic material such as blood or bone chips.

It is advantageous when the webs and meshes are surrounded on the outside by a completely closed or partially closed rim. The rim may have a peripherally uniform thickness or may vary. Handling is facilitated by the existence of a rim. Finally, when planning the implant, an implant passage can be modified, for example in the area of its wall, and can be used as a drill jig. When planning the implant, an over-correction can also be provided, viz. in such a way that the main part projects beyond the defective area, especially also in the inserted condition, so as to take into account the fact that from the main part more is degraded during healing than is new bone material built up. This serves for the purpose of achieving an esthetically appealing solution. In this way, dimples or indentations which are merely covered by skin but are visible from outside are avoided.

It has also turned out to be especially advantageous when the thickness of the webs will range from one quarter to one tenth of the length of the webs between two intersection points and/or the cross-section of the webs is designed to be a polygon or a circle or an ellipse. Especially triangular, rectangular and circular cross-sections have proven themselves. In the case of triangular cross-sections, the shape of an equilateral triangle has particularly proven itself.

In order to appropriately ensure final care of the patient, it is advantageous when at least one (through/blind) implant receiving hole for receiving an implant, such as a dental implant, is provided in the main part. So, a dental implant can already be arranged in the main part, for example, and said kit then can be inserted during an operative procedure. Of course, the invention also relates to inserting the bone augmentation piece into the bone and, resp., to inserting the kit into/onto the bone.

It is of advantage when a hole wall defining the implant receiving hole has a closed/hole-free surface. Then, proper support of e.g. a dental implant within the main part is resulting.

When the main part has at least one bone-fastening hole, viz. for receiving a fastener such as a bone screw for fixation on the bone, already initially a proper connection can be achieved between the bone augmentation piece/the bone implant and the bone. Subsequent ingrowth will further increase said initial strength.

It is worth mentioning that the main part may and shall advantageously be made up from one (single) material such as a biodegradable material such as magnesium, a magnesium alloy, iron, an iron alloy, barium or strontium, or a resorbable polymer, a ceramic material or the composites thereof such as PDLLA, PDLA, PLA, PGA, chitosan fibers/particles, HAP, CaCO₃, α-/β-TCP, hydroxyapatite and/or biphasic calcium phosphate (BCP). Of course, mixtures of said materials are also imaginable.

According to a second aspect of the invention there is provided a kit consisting of a bone augmentation piece according to the first aspect of the invention, comprising an implant inserted thereinto.

Ultimately, the invention also relates to the kit consisting of a bone augmentation piece of the type according to the invention comprising an implant inserted thereinto, such as a dental implant.

According to a third aspect of the invention there is provided a use of the bone augmentation piece according to the first aspect of the invention as a drill jig.

The invention also relates to the use of the bone augmentation piece of the type according to the invention as a drill jig.

Also, the invention relates to a medical process for inserting the bone augmentation piece of the type according to the invention onto/into a bone, e.g. of a mammal, especially a human being.

In other words, now dimensionally stable attachment and support with alloplastic material (inherent material) is made possible. In so doing, a procedure meeting the requirement is enabled, as based on the suitable dental implant position the shape and the size of the 3D construct can already initially be determined. Improvements are achieved in the field of contouring and containment. A protection against resorption is provided by membrane properties of the outer contour. This prevents ingrowth of connective tissue. Injuries of anatomic neighboring structures, especially of neighboring teeth, neighboring roots and, what is especially critical, of the nervus alveolaris inferior, are prevented. That is to say, it is possible to utilize preplanned fixation points on the remaining bone and consequently to exactly plan out the operative procedure prior to the operation.

Solid and/or porous, geometrically complex and patient-specific customized individual implants made up from bone replacement materials such as HA, α-TCP, β-TCP, BCP, magnesium or magnesium calcium zinc alloys are used and previously prepared. Manufacture at short notice of the porous, geometrically complex and patient-specific individual implant device made up from bone replacement material such as HA, α-TCP, β-TCP, BCP, magnesium and MgCaZn is enabled. An increase in strength of the produced implants can be realized by an increase in heat. Also, the manufacture of solid and/or porous geometrically complex and patient-specific individual implants made up from bone materials as resorbable bone cavity filler construct for bone regeneration is resulting. A manufacture at short notice of the porous, geometrically complex and patient-specific individual implants of bone replacement materials can be realized. The implant may consist of one or more, e.g. two, materials.

All materials can be used in the form of powder, of granulate and being present in a liquid to viscous state, wherein different quantities and material compositions can be mixed with one another.

It is finally mentioned that the bone augmentation piece may include projections, ears, extensions, tabs or plate expanding portions which may be provided on the outside, especially distally projecting so as to undergo overlapping with the adjacent bone material. In said projections, also holes may be provided to receive bone screws. Then, a plate-free connection of the bone augmentation piece to the bone can be effectuated.

Hereinafter, the invention shall be illustrated in detail by way of a drawing, wherein:

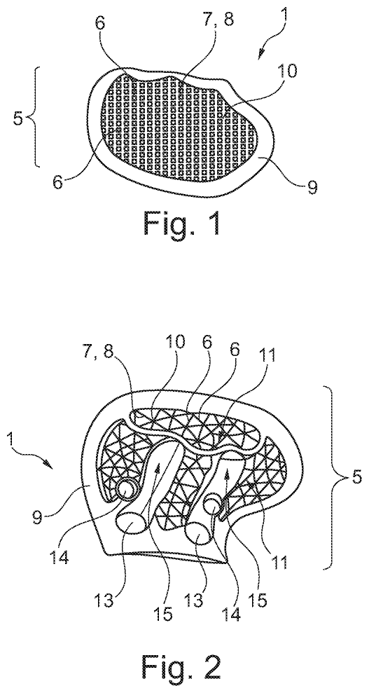

FIG. 1 shows a side view of a bone augmentation piece having a peripheral rim present in the main part within which the numerous webs thereof arranged equidistantly relative to one another form a grid structure,

FIG. 2 shows a perspective view of the side of the bone augmentation piece from FIG. 1 facing the bone which includes a pocket formed for being filled up with bone slurry/chips at the lower side of the implant and an opening provided at the top,

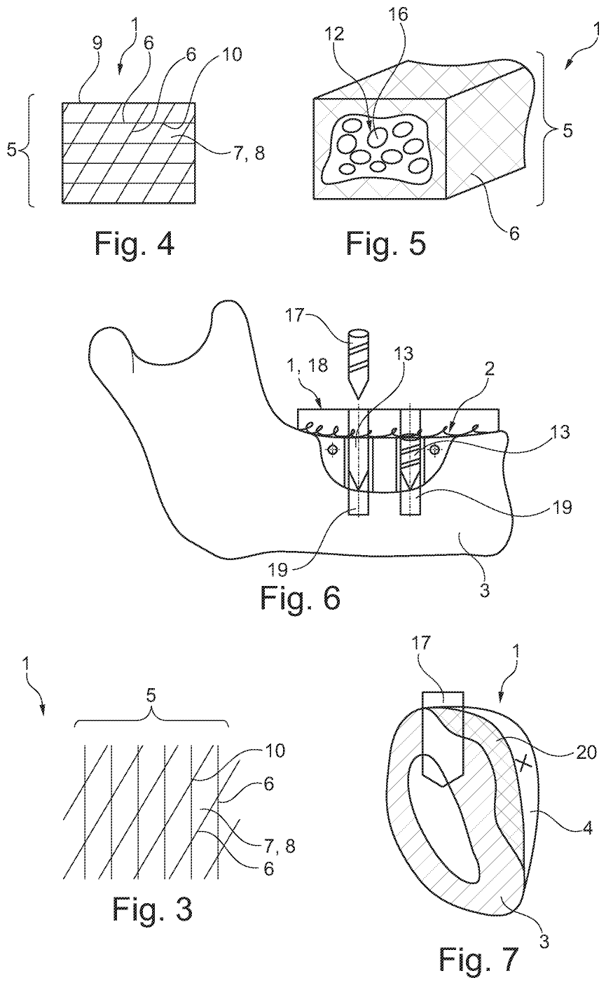

FIG. 3 shows a grid structure of the augmentation piece without a peripheral rim,

FIG. 4 shows a grid structure comparable to that of FIG. 3, however having a peripheral rim,

FIG. 5 shows a schematic structure of a pocket as shown in FIG. 2,

FIG. 6 shows the use of the bone augmentation piece as drill jig, and

FIG. 7 shows a predetermined over-correction by means of the bone augmentation piece as well as

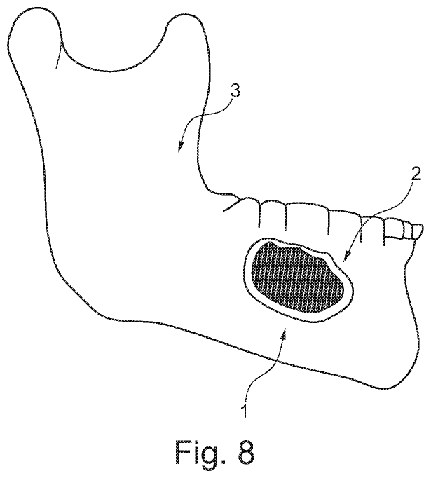

FIG. 8 shows the inserted condition of the bone augmentation piece shown in FIGS. 1 and 2 in a patient's mandible.

The figures a merely schematic. Like elements are provided with like reference numerals. Features of the individual example embodiments may be interchanged.

In FIG. 1, a bone augmentation piece 1 according to the invention is illustrated in accordance with the invention. It is a bone implant. In particular, it is a bone implant which is adapted for use in the mandible. It could also be used, after being slightly adapted, in the maxillary area.

In anticipation of FIG. 8, it is already described that the bone augmentation piece 1 fills out a defective area 2 in the bone 3. It is inserted into the defective area 2 which may be in the form of a blind hole or through-hole without a slit/without a gap. In the present case, it is flush with the surface of the bone 3. It is configured for a smooth transition into the bone 3, i.e. for abutment of the surfaces of the bone augmentation piece, on the one hand, and of the bone 3, on the other hand, viz. on the inner face and on the outer face. There may also be provided a small protrusion (either on one surface or on both surfaces), as illustrated in FIG. 7 and denoted with the reference numeral 4.

Returning to FIG. 1, it is evident that the bone augmentation piece 1 has a main part 5 made from a plurality of intersecting webs 6. The webs 6 may also be referred to as strands. A plurality of cavities 7 defining meshes 8 is provided between the webs 6.

The webs 6 are intersecting in a truss-type, e.g. rectangular, manner to create a grid structure.

As is evident from the alternative example embodiment of FIG. 2, the meshes 8 are not necessarily rectangular but may also be triangular. The main part 5 also includes, apart from the webs 6 and the meshes 8 formed by the latter, a closed rim 9. In the present case, the rim 9 has more than five times the thickness of a web 6. It is of advantage when the rim 9 has eleven times the thickness (+/−10 to 20%) of a web 6.

In FIGS. 1 and 2 intersection points 10 are referenced by way of example.

The webs 6 and the meshes 8 formed by the latter, which comprise the rim 9 if the latter is present, define a wall 11 or plural walls 11 by means of which a pocket 12 is formed.

It is striking that two implant receiving holes 13 are provided. In addition, there are also provided bone fastening holes 14. While in the implant receiving hole 13 another implant, such as a dental implant, is inserted and is fastened to the bone 3 and/or to the main part 5, a fastener such as a screw, a splint or a bolt fixing the main part 5 on the bone 3 can be introduced to the bone fastening hole 14. The bone fastening hole 14 may be arranged at least partially or even completely inside the implant receiving hole 13. Of course, it may be arranged outside thereof as well. The bone fastening hole 14 is especially preferred to partially penetrate a hole wall 15 defining the implant receiving hole 13.

In FIGS. 3 and 4, different web arrangements for a main part 5 are indicated, with a rim 9 being provided in one case (see FIG. 4) and missing in another case.

FIG. 5 illustrates a bone augmentation piece 1 comprising a main part 5 in the form of a pocket 12. Accordingly, bone chips and, resp., bone slurry 16 is/are filled into the pocket 12. Furthermore, blood products may be added.

In FIG. 3, a bone augmentation piece 1 being inserted in a defective area 2 of a bone 3 is represented. It is evident that the implant receiving holes 13 are inserted to predetermine the forced direction for a drill 17 so that the bone augmentation piece 1 acts as a drill jig 18. The countersinks 19 leading further into the bone then are utilized to be penetrated by a dental implant (not shown).

FIG. 7 illustrates the mandible as an example of a bone 3 in cross-section and an inserted bone augmentation piece 1 according to the invention is reproduced in a partially cut state. Here a protrusion 4 is provided. Said protrusion 4 is degraded, just as the remaining bone augmentation piece 1, by the body of the patient during the healing process, wherein only in the area shown in a cut state, however, which can also be defined as remaining area 20, autologous bone material is present after healing so that an esthetically appealing result is achieved.

### LIST OF REFERENCE NUMERALS

1 bone augmentation piece
2 defective area
3 bone
4 protrusion
5 main part
6 web
7 cavity
8 mesh
9 rim
10 intersection point
11 wall
12 pocket
13 implant receiving hole
14 bone fastening hole
15 hole wall
16 bone slurry/bone chip
17 drill
18 drill jig
19 countersink
20 remaining area

1. A bone augmentation piece for filling out a defective area in a bone, comprising a main part which is adapted to be inserted into the defective area without a gap, wherein the main part comprises a truss-type structure made of webs and cavities which define meshes, contains or is made up from biodegradable metallic materials and/or resorbable polymers or ceramic materials, and the webs and meshes are surrounded on the outside by a rim, the rim having more than five times the thickness of a web.

2. The bone augmentation piece 44 according to claim 1, wherein plural webs are intersecting and are interconnected at the intersection point.

3. The bone augmentation piece 44 according to claim 1, wherein the webs form a grid structure in which the majority of the meshes has an equal surface area.

4. The bone augmentation piece according to claim 3, wherein the meshes take a triangular shape or a rectangular shape.

5. The bone augmentation piece according to claim 1, wherein the webs are identical as to their geometry and/or as to the material properties.

6. The bone augmentation piece according to claim 1, wherein the webs are linear/straight or corrugated or curved.

7. The bone augmentation piece according to claim 1, wherein the webs and meshes define a wall designed in the form of a pocket.

8. The bone augmentation piece according to claim 2, wherein the thickness of the webs ranges from one quarter to one tenth of the length of the webs between two intersection points.

9. A kit consisting of a bone augmentation piece according to claim 1 comprising an implant inserted thereinto.

10. Use of the bone augmentation piece according to claim 1 as a drill jig.

11. The bone augmentation piece according to claim 2, wherein the webs form a grid structure in which the majority of the meshes has an equal surface area.
